(19) United States
(12) Patent Application Publication
(10) Pub. No.: US 2008/0241076 A1
(43) Pub. Date: Oct. 2, 2008

(54) Method and composition for increasing Erythropoietin

(75) Inventors: Shane Edward Durkee, Easton, PA (US); Gerard Dente, Cedar Grove, NJ (US)

Correspondence Address: Shane Durkee, 2374 Katie Ct., Easton, PA, 18040, US

(73) Assignees: Shane Durkee, Easton, PA (US); Gerard Dente, Cedar Grove, NJ (US)

(21) Appl. No.: 11/670,988

(22) Filed: Feb. 4, 2007

(51) Int. Cl. A61K 9/12; A61K 31/202; A61K 33/42; A61K 31/355; A61K 36/00; A61K 38/45

(52) U.S. Cl. 424/44; 514/560; 424/601; 514/458; 424/773; 424/725; 424/94.5

(57) ABSTRACT

This invention pertains to method and compositions for the purpose of increasing serum level of Erythropoietin (EPO) and the subsequent increase of red blood cells leading to an increase in athletic endurance.

### BACKGROUND OF THE INVENTION

Maintaining a high level of athletic performance is desirable in many sports and walks of life. By creating conditions that permit sustaining overall good health, training with more intensity at higher levels, and increasing training endurance for prolonged periods of time,

you increase the sense of vitality and well-being. A limiting factor in this is the amount of tissue oxygenation related to EPO production.

When production of EPO is stimulated, it binds to a specific erythropoietin receptor known as EpoR. This receptor is on the surface of red blood cell precursors found in the bone marrow. Once bound, these precursors transform into mature red blood cells (RBC) and as a result the oxygen level in blood rises. This increase in the oxygen carrying capacity results in an increase in the aerobic respiratory capacity of muscles which in turn leads to increased endurance. This increase in endurance is highly sought after by many athletes.

Endogenous EPO production is controlled first and foremost by the level of tissue oxygenation. Kidneys release EPO primarily in response to hypoxia and or anemia with the purpose of stimulating the bone marrow to produce more RBCs. EPO also causes an increase in the production of cells that mature rapidly to further accelerate RBC production. As more RBCs are produced, the total oxygen carrying capacity of the blood increases which in turn will slow down the endogenous production of EPO.

Exercise induced hypoxia has been studied as a way to stimulate endogenous production. For example, Roberts, et al, showed that only 3 minutes of exercise at maximal oxygen consumption (very intense) resulted in an increase of between 19 and 37% serum EPO 24 hours post-exercise (Roberts, D. et al. Plasma-Volume contraction and exercise induced hypoxemia modulate erythropoietin production in healthy humans. Clin Sci 2000; 98:39-45).

This invention relates a method of orally administered nutritional ingredients for the purpose of increasing the level of erythropoietin and subsequently increasing endurance.

### BRIEF SUMMARY OF THE INVENTION

The subject invention concerns method and composition for increasing the body's endogenous production of erythropoietin (EPO). More specifically, the subject invention concerns unique dietary supplementations to improve oxygen capacity and endurance to enhance the ability to prolong bouts of exercise and competitive performance. Previously, only pharmaceutical intervention was used as a means to increase EPO production and is used only as a medical treatment in individuals with disease. It may be less than ideal for Athletes and healthy normal individuals to use pharmaceutical EPO. This problem is solved with the present invention by increasing endogenous EPO production through the supplementation of dietary ingredients, most prominently, the omega 6 fatty acid Arachidonic Acid. Further compositions that compliment Arachidonic acid and support a nutritional increase of EPO are fat soluble vitamins, water soluble vitamins, minerals such as Iron, Zinc, Selenium and Cobalt. Further additions include, Phosphate and traditional herbs such as Angelica sinensis and Portulaca oleracea which are supportive of blood health and Nattokinase, a fibrinolytic agent.

### DETAILED DESCRIPTION OF THE INVENTION

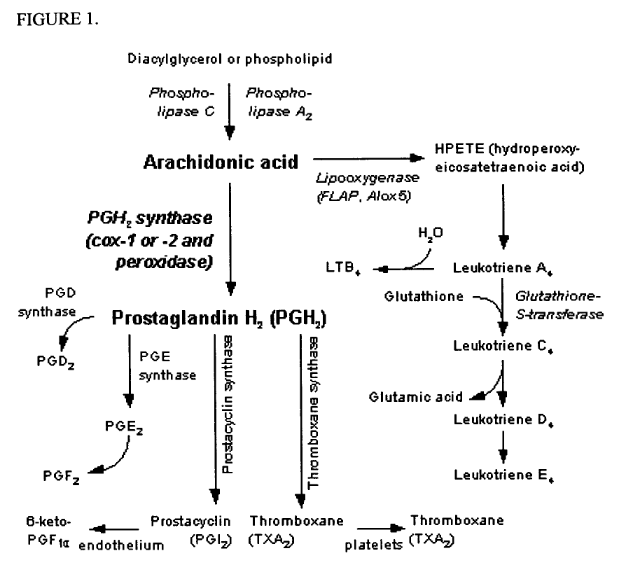

Arachidonic Acid is an omega-6 fatty acid with the chemical name all-cis-5,8,11,14-eicossatetraenoic acid. It is considered a polyunsaturated fatty acid that is present in the phospholipids of cell membranes, is abundant in the brain and is a precursor in the production of eicosanoids (see FIG. 1); in particular, prostaglandins, thromboxanes, prostacyclin and leukotrienes. It is precisely this precursor mechanism that leads to increased production of EPO. For example, several E-type prostaglandins (PGE2) produce a significant increase in iron incorporation in RBC. A study done with dog kidneys showed that both PGE2 and Arachidonic Acid (separately) increased kidney production of EPO.

The direct in-vitro conversion of Arachidonic acid to several prostaglandins was shown in a 14C-labeled Arachidonic acid study conducted by Berlin et al (Berlin, Acta Physiol Scan 1979 8; 106:441-5). The specific prostaglandins produced included PGD2, PGE2, PGF2 alpha and 6-keto-PGF1 alpha. The direct conversion of Arachidonic acid to prostaglandins was previously outlined in U.S. Pat. No. 6,841,573 to Llewellyn, where they related a method for increasing serum levels of PGf2alpha and subsequently the level of retained skeletal muscle mass.

Influence such as dietary deficiency of specific foods, micronutrients and hormone imbalances can all lead to a decrease in endogenous EPO production. Therefore it is essential to eat a healthy diet and exercise regularly for endogenous EPO production. For example, adequate protein intake is a factor in EPO production. Several animal studies have demonstrated that dietary protein adequacy and quality can affect the response to EPO. Mice that were fed a protein restrictive diet had a significant suppression in response to EPO (Depressed response of the erythropoietin splenic cell population to erythropoietin in acutely protein restricted mice. (In Vivo 1995 January-February; 9-71-73). As far as high protein intake goes, patients with various glomerular diseases, who were fed a high protein diet, have a statistically significant increase in EPO serum concentrations and urinary excretion of EPO (Rosenberg, Me. The response of erythropoietin to dietary protein in renal disease. Department of Medicine, University of Minnesota, Minneapolis 55455). Beyond protein; animal studies have also shown that calories are an important factor. Rats that have been deprived of food have a lower EPO response to hypoxia suggesting that caloric restriction induced weight loss may lead to EPO loss (Jelkmann, W. Effects of fasting on the hypoxia-induced erythropoietin production in rats. Pflugers Arch. 1983; 396:174-5). Another series of macro nutrition studies looked into the effect of glucose. Here it was shown that effects from restricted caloric diets were essentially erased when the animals had free access to a glucose solution (as drinking water). The same experiment looked at the thyroid hormone T3 and determined that a caloric deficit required significantly more T3 to maintain EPO levels (Caro, J. Erythropoietin production in fasted rats. Effects of thyroid hormones and glucose supplementation. J Lab Clin Med 1981; 98:860-8).

Minerals are essential for metabolic and physiological process in the human body. They are involved in muscle contraction, heart rhythm, oxygen transport, enzyme activation and the acid-base balance of blood, to name a few. Iron is one of several minerals that play an important factor in EPO release. To achieve effective erythropoiesis, both EPO and Iron are required (Piagnerelli, M. Iron Administration in the Critically Ill. Semin Hematol 2006; 43:S23-7). Iron itself is a component of hemoglobin, myoglobin, cytochromes and various enzymes. All of which are involved in the transport of oxygen (and metabolism of oxygen) for aerobic energy. Numerous studies have documented that athletic groups tend to be deficient in iron to the point of anemia. In fact, if an athlete becomes fatigued easily or is underperforming from a level they are used to, often they are found to be anemic (Williams, M. Dietary Supplements and Sports Performance: Minerals. Journal of the International Society of Sports Nutrition 2005; 2:43-49). Due to the importance and influence of iron. It is important to ensure dietary iron absorption. Heme iron is more easily absorbed; however supplements usually contain nonheme iron which isn't as easily absorbed. When supplementing iron, supplementation with Vitamin C as well as meat proteins can help to improve the absorption of nonheme iron. Phosphate supplementation has been used to increase erythrocyte 2,3-bisphosphoglycerate (2,3-BPG) levels which lead to an improvement in oxygen offloading from hemoglobin. Phosphate “loading” as a means to increase 2,3-BPG may improve tissue oxygenation “by inducing a rightward shift in the oxygen dissociation curve”. Studies have shown a significant increase in erythrocyte 2,3-BPG following phosphate loading. This was directly correlated to an increase in erythrocyte. It is interesting to note that unloaded subjects can see a positive correlation with phosphate supplementation as well. The authors of one particular study into phosphate loading postulate that a 30% change in 2,3-BPG increases hemoglobin P₅₀ from 25 to 28 mm Hg. This would lead to an increase in oxygenation (tissue) which is a major influence on VO₂ₘₐₓ thus resulting in performance benefits to endurance (Bremner, K. The effect of phosphate loading on erythrocyte 2,3-bisphosphoglyerate levels. Clinica Chimica Acta 2002; 323:111-114).

Zinc is a component of over 300 enzymes and involved in functions directly related to physical performance. These functions include protein synthesis and energy production in muscles. Taking that into account, it has been shown that endurance athletes that develop a zinc deficiency, can have a loss in body weight, develop fatigue and have a decrease in endurance( Williams, M. Dietary Supplements and Sports Performance: Minerals. Journal of the International Society of Sports Nutrition 2005; 2:43-49). Selenium is a component of several enzymes, most notably the enzyme glutathione peroxidase. This particular enzyme is a very important cellular enzyme that acts as an antioxidant. It has been theororized that selenium supplementation could help in the prevention of RBC peroxidation (Williams, M. Dietary Supplements and Sports Performance: Minerals. Journal of the International Society of Sports Nutrition 2005; 2:43-49). When you take into account the fact that kidneys accumulate the highest levels of selenium and that they are the major source of plasma glutathione peroxidase, this theory starts to come together. For example, decreased blood selenium levels and glutathione peroxidase activity are markedly decreased in cases of chronic renal failure. When these patients are supplemented with both EPO and selenium, there is a significant increase in the elemental concentration found in both whole blood and plasma (Adamowicz, A. Effect of erythropoietin therapy and selenium supplementation on selected antioxidant parameters in blood of uremic patients on long-term hemodialysis. Med Sci Monit 2002; 8:CR202-205)

In addition to minerals, several vitamins may have affects on EPO. Vitamin A, as an antioxidant, has been shown in animal studies to increase renal EPO production. The authors of this study postulate that decreases in reactive oxygen species by specific antioxidants, such as vitamin A, feed into the mechanism that regulates renal synthesis of EPO (Neumcke, I. Effects of Pro and Antioxidative Compounds on Renal Production of Erythropoietin. Endocrinology 1999; 140:641-645). Vitamin E, also an antioxidant, has been shown to stabilize endogenous glutathione. The B-Vitamins Folic Acid, B6, and B12 all play numerous roles in the body including roles associated with haemesynthesis, haematopoeisis. Formulation targeted towards increasing endogenous production of EPO may want to include all three of these B-Vitamins.

Vitamin C may be involved in several phases of iron transport. For example, Vitamin C has a role in mobilizing iron, maintaining iron in a reduced form, which stimulates the production of ferritin, and it potentiates the incorporation of reduced iron into protoporphyrin (this combination forms the heme group in hemoglobin and myoglobin). In hemodialysis patients vitamin C supplementation has resulted in significant increases in hemoglobin levels (Keven, K. Randomized, crossover study of the effect of vitamin C on EPO response in hemodialysis patients. Am J Kidney Dis. 2003; 42:848-849). In a separate study, Vitamin C was shown to improve responsiveness to EPO by its antioxidant effects and by augmenting Iron mobilization (Attallah, N. Effect of intravenous ascorbic acid in hemodialysis patients with EPO hyporesponsive anemia and hyperferritinemia Am J Kidney Dis. 2006; 47:683-5).

The amino acid Carnitine is derived from the dietary sources, meat and dairy. It can also be synthesized endogenously from a combination of the essential amino acids lysine and methionine in both the liver and the kidney. Although most widely known for its effects on heart muscle and fat oxidation, one study showed an increase in erythrocyte survival time with Carnitine supplementation (Mechanick, J. American Association of Clinical Endocrinologists Medical Guidelines for the Clinical use of Dietary Supplements and Nutraceuticals. Endocrine Practice 2003; 9:441-443).

There are various herbs that are considered to be blood tonics. There are at least two herbs with some research conducted in relation to EPO, Angelica sinensis and Portulaca oleracea. Chinese researchers were able to reduce the amount of rEPO needed by hemodialysis patients, simply by supplementing the patients with Angelica sinensis (Bradely, R. R. Hematopoietic effect of Radix angelicae sinensis in a hemodialysis patient.

AM J Kidney Dis. 1999; 34:349-54). In an animal study, a different group of Chinese researchers increased the survival time of mice in hypoxic conditions by supplementation with flavones extracted from Portulaca oleracea. The authors concluded that these flavones may improve the expression level of EPO and accerlate the generation of erythrocyte and hemoglobin (Dong, L. W. Effects of flavones extracted from Portulaca oleracea on ability of hypoxia tolerance in mice and its mechanism. Department of Military Hygiene, Second Military Medical University, Shanghai).

Blood Volume and Sodium play a factor in the increase (or decrease) of EPO levels. Two important components of blood are plasma (the aqueous portion) and erythrocytes (RBC) which have the primary responsibility for oxygen transport. Sodium is required by all cells to maintain normal fluid balance. When sodium levels are not in balance (what's taken in and what is excreted) the volume of water in the blood will change. When there is more sodium taken in than excreted, blood volume will increase as fluid is retained. Exactly the opposite happens when the level of sodium being taken in decreases. The blood volume will lower and with it levels of EPO lower.

Nattokinase is a potent fibrinolytic enzyme that can help keep blood circulation normal and dissolve clots. Nattokinase is extracted from a Japanese food called Natto and its fibrinolytic properties were first discovered by Dr. Sumi in 1980. When strands of fibrin accumulate in a blood vessel, a blood clot (or thrombi) is formed. If blood flow is eventually blocked, the result can be angina and heart attacks. Nattokinase has been shown to be efficacious in preventing coagulation of blood and in dissolving blood clots (Heissler, M. The Japanese Biotechnology Industry Biotechnology and Development Monitor 1995; 22:5-6). Nattokinase has been the subject of several studies including human trials. In some of the early animal studies; dogs, with induced clots, who received Nattokinase regained normal blood circulation within five hours of treatment. Another animal study showed a 62% regain in blood flow with Nattokinase compared to a 15.8% increase with plasmin which is the body's natural fibrinolytic enzyme. Human trials showed that feeding Nattokinase to individuals generated a heightened ability to dissolve blood clots and this ability was retained for 2 to 8 hours.

What is claimed is:

1. A method for increasing serum level of Erythropoietin (EPO) where in said method comprises administering orally a composition comprised of Arachidonic Acid.

2. A method, according to claim 1, where in said composition is comprised of a precursor (an Omega 6 fatty acid) to Arachidonic acid.

3. The method, according to claim 1, wherein said composition further comprises minerals including Iron, Phosphate, Zinc, Selenium and Cobalt.

4. The method, according to claim 2, where in said composition further encompasses a phase in period of phosphate loading, where said phosphate is orally administered at a higher concentration than Arachidonic acid for a period of 1 to 15 days.

5. The method, according to claim 1, where in said composition further comprises fat soluble vitamins including Vitamin A, and Vitamin E.

6. The method, according to claim 1, where in said composition further comprises water soluble vitamins including Vitamin B6, Vitamin B12, Folic Acid and Vitamin C.

7. The method, according to claim 1, where in said composition further comprises cationic and dibasic amino acids.

8. The method, according to claim 1, where in said composition further comprises the branched-chain amino acids (Leucine, Valine and Iso-Leucine)

9. The method, according to claim 1, where in said composition further comprises the addition of Angelica Sinesis

10. The method, according to claim 1, where in said composition further comprises the addition Portulac Oleracea

11. The method, according to claim 1, where in said composition further comprises Nattokinase.

12. The method, according to claims 1, 2, 3, 4, 5, 6, 7, 8, 9 where in said composition is administered orally by a gelatin capsule(s) or tablet(s).

13-14. (canceled)

15. The method, according to claims 1, 2, 3, 4, 5, 6, 7, 8, 9 where in said composition is administered orally by a protein enriched product.

16. The method, according to claims 1, 2, 3, 4, 5, 6, 7, 8, 9 where in said composition is administered orally by a powdered or compressed effervescent formulation.

17. The method, according to claims 1, 2, 3, 4, 5, 6, 7, 8, 9 where in said composition is administered in conjunction with a high intensity, short duration endurance exercise.

18. The method, according to claims 1, 2, 3, 4, 5, 6, 7, 8, 9 where in said composition results in an increase in endogenous erythropoietin levels.

19. The method, according to claims 1, 2, 3, 4, 5, 6, 7, 8, 9 where in said composition results in an increase in athletic endurance.
